(19) United States
(12) Patent Application Publication
(10) Pub. No.: US 2024/0417355 A1
(43) Pub. Date: Dec. 19, 2024

(54) PROCESS FOR THE HYDROFORMYLATION OF DIISOBUTENE WITH PRECEDING DISTILLATION

(71) Applicant: Evonik Oxeno GmbH & Co. KG, Marl (DE)

(72) Inventors: Peter Kucmierczyk, Herne (DE); Robert Franke, Marl (DE); Anna Chiara Sale, Duesseldorf (DE); Dirk Fridag, Haltern am See (DE); Ana Markovic, Haltern am See (DE); Armin Matthias Rix, Marl (DE); Maren Termühlen, Dortmund (DE); Laura-Selin Harding, Dorsten (DE); Jan Benedikt Metternich, Recklinghausen (DE)

(73) Assignee: Evonik Oxeno GmbH & Co. KG, Marl (DE)

(21) Appl. No.: 18/739,599

(22) Filed: Jun. 11, 2024

(30) Foreign Application Priority Data
Jun. 14, 2023 (EP) 23179204.5

(51) Int. Cl. C07C 45/50; B01D 3/14; B01J 29/70; B01J 31/08; B01J 31/24; C07C 45/78; C07C 45/83

(52) U.S. Cl. CPC C07C 45/505; B01D 3/143; B01J 29/7007; B01J 31/08; B01J 31/24; C07C 45/786; C07C 45/83; B01J 2231/321; B01J 2231/52; B01J 2531/822

(57) ABSTRACT

A process is developed for the hydroformylation of diisobutene, in which the diisobutene stream used is subjected to a distillation prior to the hydroformylation in order to enrich 2,4,4-trimethylpent-1-ene in the stream to be hydroformylated. The hydroformylation is carried out with synthesis gas in the presence of a homogeneous catalyst system that comprises at least Co or Rh and optionally a phosphorus-containing ligand.

### CROSS-REFERENCE TO RELATED APPLICATIONS

This patent application is based on and claims priority pursuant to 35 U.S.C. § 119 (a) to European Patent Application No. 23179204.5, filed on Jun. 14, 2023, in the European Patent Office, the entire disclosure of which is hereby incorporated by reference herein.

### BACKGROUND

### Field of the Invention

The present invention relates to a process for the hydroformylation of diisobutene, in which process the diisobutene stream used is subjected to a distillation prior to the hydroformylation in order to enrich 2,4,4-trimethylpent-1-ene in the stream to be hydroformylated. The hydroformylation is carried out with synthesis gas in the presence of a homogeneous catalyst system that comprises at least Co or Rh and optionally a phosphorus-containing ligand.

### Description of Related Art

Diisobutene is a technically relevant product obtained by dimerization of isobutene. Diisobutene consists of the isomers 2,4,4-trimethylpent-1-ene (hereinbelow also: TMP1) and 2.4,4-trimethylpent-2-ene (hereinbelow also: TMP2) with a mass distribution of TMP1:TMP2 of about 78:22 to 81:19 (equilibrium distribution). This mixture can inter alia be converted into higher-value products in carbonylation processes. Particularly in carbonylation processes, such as methoxycarbonylation (reaction product here is methyl 3,5,5-trimethylhexanoate) or hydroformylation (reaction product here is 3,5,5-trimethylhexanal), the internal olefin TMP2 has significantly lower reactivity than the terminal olefin TMP1.As a consequence of inadequate stability of the catalysts or economic factors (for example the size of the reactor), it is often not possible to adjust the reaction conditions or residence times in these carbonylation reactions sufficiently to allow the terminal TMP2 to react to completion.

### SUMMARY OF THE INVENTION

It is accordingly an object of the present invention to provide a process for the hydroformylation of streams which does not exhibit the aforementioned problems. In particular, the hydroformylation is to be carried out with diisobutene streams that have a high proportion of 2,4,4-trimethylpent-1-ene.

This object was achieved by the process of the invention according to the description herein. Preferred embodiments are also specified.

### BRIEF DESCRIPTION OF THE DRAWINGS

FIG. 1 shows an embodiment in which the diisobutene stream (1) is supplied to the distillation column (2) in (a).

FIG. 2 shows a further embodiment in which an isomerization (12) is additionally included.

### DETAILED DESCRIPTION OF THE INVENTION

The present invention describes a process for the hydroformylation of diisobutene, wherein the process comprises at least the following steps:
a. supplying a diisobutene stream comprising 2,4,4-trimethylpent-2-ene and 2,4,4-trimethylpent-1-ene to at least one distillation column and separating the diisobutene stream into at least one overhead stream that is enriched with 2,4,4-trimethylpent-1-ene compared to the diisobutene stream used and contains more than 85% by weight of 2,4,4-trimethylpent-1-ene, and a residual stream depleted in 2,4,4-trimethylpent-1-ene compared to the diisobutene stream used;
b. supplying the overhead stream to the hydroformylation whereby 2,4,4-trimethylpent-2-ene and 2,4,4-trimethylpent-1-ene are in a reaction zone reacted with synthesis gas in the presence of a homogeneous catalyst system comprising at least Co or Rh and optionally a phosphorus-containing ligand to obtain a liquid product mixture, the liquid product mixture comprising at least the aldehydes formed by the hydroformylation, the homogeneous catalyst system and unreacted 2,4,4-trimethylpent-2-ene and 2,4,4-trimethylpent-1-ene;
c. removing the homogeneous catalyst system from the liquid product mixture to obtain a crude product mixture comprising at least the aldehydes formed by the hydroformylation and unreacted 2,4,4-trimethylpent-2-ene and 2,4,4-trimethylpent-1-ene;
d. distillative processing of the crude product mixture in at least one distillation column to remove unreacted 2,4,4-trimethylpent-2-ene and unreacted 2,4,4-trimethylpent-1-ene, wherein the unreacted 2,4,4-trimethylpent-2-ene and 2,4,4-trimethylpent-1-ene are removed and recycled to the hydroformylation in step b.

An advantage of the process is the distillation of the diisobutene stream before the hydroformylation, since this makes it possible to increase the proportion of 2,4,4-trimethylpent-1-ene in the overhead stream of the at least one distillation column compared to the stream used. Thus, it is possible to achieve proportions of more than 85% by weight, preferably more than 90% by weight, more preferably more than 95% by weight, of 2,4,4-trimethylpent-1-ene in the stream, which is advantageous for the downstream hydroformylation for the reasons mentioned (higher reactivity of 2,4,4-trimethylpent-1-ene).

Step a of the process of the invention relates to the distillation of a diisobutene stream comprising 2,4,4-trimethylpent-2-ene and 2,4,4-trimethylpent-1-ene. The diisobutene stream is separated in this step into at least one overhead stream enriched with 2,4,4-trimethylpent-1-ene compared to the diisobutene stream used that preferably contains more than 85% by weight of 2,4,4-trimethylpent-1-ene, further preferably more than 90% by weight of 2,4,4-trimethylpent-1-ene, particularly preferably more than 95% by weight of 2,4,4-trimethylpent-1-ene, and a residual stream depleted in 2,4,4-trimethylpent-1-ene compared to the diisobutene stream used.

The diisobutene stream used in step a comprises 2,4,4-trimethylpent-2-ene and 2,4,4-trimethylpent-1-ene. Such streams may be diisobutene streams which can be produced by dimerization from isobutene or isobutene-containing hydrocarbon mixtures, for example those disclosed in EP 1 360 160 B1. In addition, the streams to be used here may occur as unreacted residual streams in carbonylation processes, for example in a methoxycarbonylation or in a hydroformylation. It has already been mentioned that the input stream can be supplied to the isomerization and/or to the distillation. The designation of steps a. and b. in the present description therefore does not constitute a prioritization or chronology. It will be apparent that both steps must be carried out and that the individual steps are fed from the respective other step. Which step is therefore considered to be carried out first is therefore of secondary importance.

The distillation in step a of the process of the invention is carried out in at least one distillation column. Distillation columns are generally known to those skilled in the art. The distillation unit of the present invention comprises preferably at least one distillation column, more preferably at least two distillation columns. The following description of features of the distillation column also applies whenever there is more than one distillation column in the distillation unit.

The at least one distillation column preferably has internals to cope with the separation task. Appropriate internals are familiar to those skilled in the art. Particularly suitable here are random or structured packings, such as those known to those skilled in the art under trade names such as MellaPak®,

MellapakPlus®, Flexipac®, etc. In a preferred embodiment of the present invention, the at least one distillation column comprises at least 50 theoretical plates, preferably at least 70 theoretical plates, more preferably at least 90 theoretical plates. If two or more distillation columns are present, the distillation columns may have an identical or different number of theoretical plates.

The operating parameters of the at least one distillation column are oriented to the separation task and the design of the distillation column. In the present process, it is preferable that the at least one distillation column is operated at reduced pressure, particularly preferably at a pressure of 0.2 to 0.9 bar. In the context of the present invention, reduced pressure is present whenever operations are carried out below the ambient pressure of approx. 1 bar, i.e. the atmospheric pressure present at the respective site. If there are two or more distillation columns, the distillation columns may be operated at the same or different pressure.

It is additionally preferable that the temperature in the bottoms of the at least one distillation column is in the range from 50 to 100° C. If there are two or more distillation columns, the distillation columns may be operated at the same or different temperature.

A further parameter for the design of distillation columns is the reflux ratio. The reflux ratio means the ratio of reflux (recyclate) to distillate (withdrawn condensate, in this case therefore the overhead stream K). The reflux is thus a condensed portion of the overhead stream that is returned to the distillation column. It is preferable in accordance with the invention that the reflux ratio of the at least one distillation column in the distillation in step a. is in the range from 5 to 15.

In the at least one distillation column, the distillation according to the invention in step a gives rise to an overhead stream that preferably contains at least 85% by weight of 2,4,4-trimethylpent-1-ene, further preferably at least 90% by weight of 2,4,4-trimethylpent-1-ene, particularly preferably at least 95% by weight of 2,4,4-trimethylpent-1-ene. In addition, the distillation affords a residual stream depleted in 2,4,4-trimethylpent-1-ene compared to the diisobutene stream used.

The residual stream additionally obtained in the distillation in step a can be withdrawn as a bottoms stream or as a side stream of the at least one distillation column. If there are two distillation columns, the residual stream is obtained in the final distillation column, irrespective of whether it is withdrawn as a side stream or bottoms stream. If the residual stream can be withdrawn as a side stream, this may in principle be arranged at the same height or below the inlet for the employed diisobutene stream. It will be apparent here that the inflow and outflow must be arranged an adequate distance apart. The residual stream, irrespective of whether it is withdrawn as a side stream or bottoms stream, preferably contains 80% to 92% by weight of 2,4,4-trimethylpent-2-ene.

If the residual stream in the distillation in step a is withdrawn as a bottoms stream of the at least one distillation column, a purge stream comprising high-boilers formed during the isomerization, for example dimers or oligomers of 2,4,4-trimethylpent-2-ene and/or 2,4,4-trimethylpent-1-ene, is preferably withdrawn from the residual stream. The removed high boilers can be incinerated to generate energy or can undergo hydrogenation to produce valuable alkanes.

If the residual stream in the distillation in step a is withdrawn as a side stream of the at least one distillation column, a stream comprising high-boilers formed during the isomerization, for example dimers or oligomers of 2,4,4-trimethylpent-2-ene and/or 2,4,4-trimethylpent-1-ene, is preferably obtained in the bottoms of the at least one distillation column. The removed high boilers can be incinerated to generate energy or can undergo hydrogenation to produce valuable alkanes.

Distillation columns are, as is known, heated in the bottoms to cope with the separation task. It is possible here to supply energy via heating steam, which is often available at chemical production sites. This is done by passing a portion of the bottoms through a heat exchanger (reboiler), heating it there and then returning it to the bottoms of the distillation column. To reduce energy requirements and/or CO2 emissions, the distillation in step a may be designed with thermal integration. Thermal integration means that energy produced or present within the process is used elsewhere. In the present case, the (released) condensation energy arising at the top of at least one distillation column of the distillation unit is particularly suitable. In a preferred embodiment of the present invention, thermal integration is carried out in step a. in such a way that at least part of the condensation energy at the top is used for heating the bottoms.

Another option for thermal integration is what is known as vapour compression, in which at least a portion of the vapour (overhead stream) is compressed, i.e. pressurized to a higher level and optionally heated.

The vapour thus compressed and optionally heated is supplied to the heat exchanger (reboiler) in order to heat the bottoms. This makes use of the heat of condensation of the vapour.

A further option is the use of a heat pump. Heat pumps are operated using a working medium such as n-butane or water. The condensation energy is in this case thus first transferred in a heat exchanger to a working medium and from there, in a further suitable heat exchanger, to the distillation bottoms. The working medium is here usually conveyed via a compressor to the heat exchanger in the bottoms. It is in principle also possible to employ two-stage or multistage heat pumps that have more than one compressor stage. In the case of a two-stage heat pump, there is not just one working medium, but two working media, wherein an exchange of energy between the first and the second working medium also takes place in a heat exchanger. In the case of multistage configurations, correspondingly more working media are present.

The overhead stream obtained from the distillation in step a is supplied to the subsequent step b of hydroformylation and reacted there. In a preferred embodiment, the residual stream also obtained from the distillation in step a is supplied to an isomerization in which 2,4,4-trimethylpent-2-ene is at least partially isomerized to 2,4,4-trimethylpent-1-ene using a heterogeneous catalyst based on a zeolite or on an ion-exchange resin. The isomerization can thus be carried out with the residual stream depleted in 2,4,4-trimethylpent-1-ene compared to the diisobutene stream used in step a and withdrawn from the distillation in step a.

The performance of an isomerization prior to the distillation in step a has the advantage that a heterogeneous catalyst system is used. Such a catalyst system does not have to be removed from the isomerization stream and remains in the reaction vessel/reactor. The catalyst systems based on a zeolite or on an ion-exchange resin that are preferred in accordance with the invention additionally permit high conversions to be achieved.

The isomerization can in principle be carried out in any suitable reactor. It is possible for the isomerization to take place in a single reactor or in two or more reactors connected in parallel or in series. Execution in batches or in continuous operation is also possible. The isomerization is preferably carried out in one or more continuously operated reactors that are customarily employed in solid/liquid contact reactions. When using continuously operated flow reactors, a fixed bed is usually, but not always, employed. When a fixed-bed flow reactor is used, the liquid can flow in an upward or downward direction. In most cases, downward flow of the liquid is preferable. In addition, it is possible to operate the reactor with product recycling or in straight pass. A concept different to that of fixed-bed reactors is for example reactors in which the ion exchanger or zeolite is suspended in a liquid phase.

The reactors used for the isomerization may be tubular reactors or tube bundle reactors, especially ones having internal tube diameters of 10 to 60 mm. The length-to-diameter ratio of the catalyst bed may be varied here, either by the geometric dimensions of the reactor or by its filling level. At the same amount of contact and load (LHSV), it is thus possible to achieve different superficial velocities and to selectively influence the heat transfer to the cooling medium.

The cooling of the tubes of the reactor, whether it be a tubular reactor or a tube bundle reactor, can be effected via a cooling medium (for example cooling water or a heat-absorbing process fluid for thermal integration) via the shell space of the reactor or a heat exchanger in an external recycling system. Especially when using liquid heating media, the shell side is constructed such that the temperature gradient in contact with all tubes is as homogeneous as possible. The technical measures necessary for this are known to those skilled in the art and are described in the literature (installation of baffle plates, disc-on-donut construction, infeed/outfeed of heat-transfer medium at various points in the reactor, etc.). Preferably, the reaction medium and heat transfer medium are respectively conveyed through the reactor tubes and reactor jacket in cocurrent flow, more preferably from top to bottom. A preferred embodiment is described for example in DE 10 2006 040 433 A1.

The isomerization of 2,4,4-trimethylpent-2-ene to 2,4,4-trimethylpent-1-ene takes place exothermically, i.e. it proceeds with the release of energy, which results in warming of the reaction mixture. In order to limit the temperature rise, it is possible to dilute the input stream, for example by recycling the product.

The reactor(s) used in the isomerization may be operated adiabatically, polytropically or practically isothermally. Practically isothermally means that the temperature is at no point in the reactor more than 10° C. higher than the temperature at the reactor entrance. In the case of adiabatic operation of the reactors, it is usually advantageous to arrange a plurality of reactors in series and to provide cooling between the reactors. Reactors that are suitable for polytropic or practically isothermal operation are for example the tube bundle reactors already mentioned and also stirred-tank reactors and loop reactors.

The process can be executed at rather mild temperatures. The isomerization is preferably carried out at a temperature of from 25 to 90° C., preferably 30 to 80° C., more preferably 35 to 70° C. In addition, the isomerization of the invention can be carried out at a pressure equal to or greater than the vapour pressure of the input stream mixture and/or of the reaction mixture at the respective reaction temperature, preferably at a pressure of more than 0 bar but less than 40 bar. Further preferably, the isomerization is carried out in the liquid phase. It should be clear that in this case the pressure and temperature must be chosen such that the input stream is present, or may be present, in the liquid phase.

In the reactor(s), it is also possible to use various catalysts based on a zeolite or on an ion-exchange resin for the isomerization. For example, a mixture of ion-exchange resins of different reactivity may be used. It is likewise possible for a reactor to contain catalysts of different activities that are arranged for example in layers. If more than one reactor is used, the individual reactors may be filled with the same or different catalyst(s) based on a zeolite or on an ion-exchange resin.

As a heterogeneous catalyst, a catalyst based on a zeolite or on an ion-exchange resin may be used for the isomerization. Suitable zeolites and ion-exchange resins are widely available on the market. It has been found that the catalyst based on a zeolite preferably has a Si: Al ratio in the range from 40:1 to 200:1. In the case of catalysts based on an ion-exchange resin, preferably the styrene-divinylbenzene type as the H-form and in a partially neutralized form has in addition been found to be highly suitable.

When the catalyst is a zeolite, some zeolites have proven to be particularly advantageous, for example beta-and gamma-zeolites. The zeolite is therefore preferably selected from the group consisting of Z-beta-H-25, Z-beta-H-38, Z-beta-H-360, Z-Mor-H-20, Z-Y-H-60, Z-Y-H-80, Z-beta-H, Z-CFG-1, Z-beta-ammonium-38, Z-CBV 760 CY (1.6), Z-CBV 780 CY (1.6), CP 814E CY (1.6), CBV 500 CY (1.6), H-CZB-150 and mixtures thereof.

lon-exchange resins that may be used include for example ion-exchange resins produced by the sulfonation of phenol/aldehyde condensates or by the sulfonation of copolymers of aromatic vinyl compounds. Examples of aromatic vinyl compounds for the production of the copolymers are: styrene, vinyltoluene, vinylnaphthalene, vinylethylbenzene, methylstyrene, vinylchlorobenzene, vinylxylene and divinylbenzene. Particular preference is given to using for the isomerization ion-exchange resins produced by the sulfonation of copolymers formed by the reaction of styrene with divinylbenzene. The ion-exchange resins may be produced in gel-like, macroporous or sponge-like form. The properties of these resins, in particular specific surface area, porosity, stability, swelling or shrinkage and exchange capacity may, as is known, be varied via the production process.

lon-exchange resins of the preferred styrene-divinylbenzene type are sold inter alia under the following trade names: CT 151 and CT275 from Purolite, Amberlyst® 15, Amberlyst® 35, Amberlite® IR-120, Amberlite® 200 from Rohm&Haas, Dowex M-31 from Dow, Lewatit® K 2621, Lewatit® K 2431 from Lanxess.

The pore volume of the ion-exchange resins employable as catalysts, in particular those of the preferred styrene-divinylbenzene type, is preferably 0.3 to 0.9 ml/g, more preferably 0.5 to 0.9 ml/g. The pore volume can be determined for example by adsorptive techniques. The particle size of the ion-exchange resins is preferably from 0.3 mm to 1.5 mm, more preferably 0.5 mm to 1.0 mm. A narrower or broader particle size distribution may be chosen. It is thus possible for example to use ion-exchange resins having a very uniform particle size (monodisperse resins).

The ion-exchange resins employable as catalysts for the isomerization may be present as partially neutralized ion-exchange resins. For this purpose, the ion-exchange resin may be treated with acids or bases, as described in EP 1 360 160 B1.

The optional isomerization affords an isomerization stream in which the proportion of 2,4,4-trimethylpent-2-ene is lower and the proportion of the 2,4,4-trimethylpent-1-ene in the isomerization stream higher than in the residual stream used. The isomerization stream may be supplied at least in part to the distillation in step a, preferably the entire isomerization stream is supplied to the distillation in step a.

The process may also include the supply of an input stream with which the process is fed. The input stream with which the process is fed and which comprises 2,4,4-trimethylpent-2-ene and 2,4,4-trimethylpent-1-ene can be conducted to the distillation unit in step a in addition to the isomerization stream and/or to the isomerization in addition to the residual stream.

The flexible supply of the input stream is advantageous here. This allows the process to be adapted to the respective conditions, for example according to the input stream that is employed. If the proportions of 2,4,4-trimethylpent-2-ene and 2,4,4-trimethylpent-1-ene in the input stream are close to the equilibrium distribution, the stream can be supplied directly to the distillation. If the proportions of 2,4,4-trimethylpent-2-ene and 2,4,4-trimethylpent-1-ene in the input stream differ from the equilibrium distribution, the stream could initially be supplied to the isomerization. Incorporation into existing production plants is thus possible in a straightforward manner according to the composition of the input stream.

The overhead stream obtained from the distillation in step a is, as mentioned, supplied to the subsequent step b of hydroformylation and reacted there.

The diisobutenes, i.e. 2,4,4-trimethylpent-2-ene and 2,4,4-trimethylpent-1-ene, are in step b reacted with synthesis gas (mixture of carbon monoxide (CO) and hydrogen (H₂)) to form an aldehyde. This increases the number of carbon atoms in the aldehyde, compared to the diisobutene used, by 1 carbon atom. The diisobutenes (8 carbon atoms) accordingly give rise to an aldehyde having 9 carbon atoms, namely trimethylhexanal.

The synthesis gas for the process according to the invention may be employed in different mixing ratios of carbon monoxide and hydrogen. The molar ratio between synthesis gas and the employed hydrocarbon stream containing the olefins to be hydroformylated should be between 6:1 and 1:1, preferably between 3:1 and 1:1, particularly preferably between 2:1 and 1:1. The hydroformylation may optionally be performed in the presence of an additional solvent known to those skilled in the art, though it is preferable when no additional solvent is used and the employed olefin functions as solvent in the hydroformylation.

The homogeneous catalyst system employable in the hydroformylation may contain Co or Rh, preferably Rh, and optionally a phosphorus-containing ligand. Corresponding catalyst systems are familiar to those skilled in the art. The use of a phosphorus-containing ligand is preferred.

In a particularly preferred embodiment the homogeneous catalyst system comprises or consists of Rh and a phosphorus-containing ligand. Suitable ligands for the catalyst systems according to the invention are known to those skilled in the art. The phosphorus-containing ligand for the catalyst system according to the invention is preferably a phosphine (for example TPP (triphenylphosphine), a monophosphite (for example Alkanox 240 (tris (2,4-di-tert-butylphenyl) phosphite) or a bisphosphite (for example biphephos). It is also possible to employ mixtures of ligands.

The temperature during the homogeneously catalyzed hydroformylation is preferably in the range from 80° C. to 250° C., further preferably in the range from 90°° C. to 225° C. and particularly preferably in the range from 100° C. to 210° C. The pressure during the homogeneously catalyzed hydroformylation is preferably in the range from 100 to 350 bar, further preferably in the range from 175 to 325 bar and particularly preferably in the range from 200 to 300 bar.

The pressure during the hydroformylation typically corresponds to the total gas pressure. In the context of the present invention the total gas pressure is to be understood as meaning the sum of the prevailing pressures of all present gaseous substances, i.e. the pressure of the (total) gas phase. In the present process this especially corresponds to the sum of the partial pressures of CO and H₂, i.e. the total gas pressure is then the synthesis gas pressure.

Homogeneously catalyzed hydroformylations may be operated as liquid output processes (“liquid recycle”) or as gas output processes (“gas recycle”). Both process variants are known to those skilled in the art and described in many textbooks. A specific selection of such a process is unnecessary in the context of the present invention because the process may in principle be performed in both ways. What is in any case important in homogeneous catalysis is the separation of the catalyst system from the reaction output. In the case of a liquid output this is possible for example via flash processes or membrane separation. In the case of a gaseous output for example by condensation and/or scrubbing. This too is known to those skilled in the art and does not require comprehensive explanation. The further workup of the reaction output, in particular the separation of the reaction product, is likewise familiar to those skilled in the art and may be carried out for example by means of a thermal separation process such as distillation. Thermal separation/thermal separation processes in the context of the present invention is to be understood as meaning separation processes where separation is effected by means of the boiling point.

The described hydroformylation in step b affords a liquid product mixture that comprises at least the aldehyde formed by the hydroformylation, the homogeneous catalyst system and unreacted diisobutenes 2,4,4-trimethylpent-2-ene and 2,4,4-trimethylpent-1-ene.

The product mixture thus obtained is supplied to the following step c in order to remove the homogeneous catalyst system from the liquid product mixture. Before the product mixture is fed in, low-boiling components, for example low-boiling by-products, can be removed, for example by thermal separation (flash, distillation or the like), for which depressurization of the highly pressurized product mixture may be required.

A preferred embodiment of the present invention further comprises a cooling of the product mixture before the membrane separation in step c to a temperature between 40° C. and 100° C., preferably between 50° C. and 95° C., particularly preferably between 60° C. and 90° C. This requires a suitable cooling apparatus. The cooling is especially carried out in an output cooler. Apparatuses that have proven suitable include for example tube bundle heat exchangers, wherein the reaction mixture is preferably passed through the tubes and the cooling medium is preferably passed through the shell of the heat exchanger.

The cooling of the product mixture ensures a reduction in the catalyst metal usage factor. The problem is that during a hydroformylation and the subsequent separation a small portion of the metal, in particular of the rhodium, is always lost in various ways. Due to the high prices of the metals to be employed, in particular of the metal, this increases process costs because the losses have to be compensated by replenishment. However, the cooling according to the invention has the effect that the usage factor falls, i.e. less catalyst metal, in particular rhodium, is lost and thus less is required for replenishment. This accordingly makes it possible to considerably reduce process costs.

The removal of the homogeneous catalyst system to obtain the crude product mixture in step c may be effected with the aid of various separation processes, for example by thermal separation and/or by membrane separation. Suitable processes are familiar to those skilled in the art. It is preferable when initially a thermal separation, for example an evaporation, and subsequently a membrane separation are carried out. In the evaporation it is mainly product aldehydes (trimethylhexanal) and unconverted diisobutenes that are obtained overhead as crude product mixture. A high boiler phase containing the homogeneous catalyst, trimethylhexanal and any high boilers formed is obtained in the bottoms. The high boiler phase may then be subjected to a membrane separation to discharge any high boilers. As is known, a membrane separation gives rise to a retentate and a permeate. The permeate may be sent for further processing.

The retentate comprises the homogeneous catalyst system. It is preferable in accordance with the invention that the retentate is recycled to the hydroformylation in step b/to the reaction zone where the hydroformylation is carried out. This allows the catalyst system to be reused. In the preferably continuous execution of the described process, this gives rise to a catalyst cycle in which at most only minor process-related catalyst losses need to be compensated. Where the diisobutene stream and the homogeneous catalyst system are, in accordance with the preferred embodiment, mixed more particularly in a suitable mixing vessel prior to the hydroformylation in step b, the retentate is supplied to the mixing vessel.

Any suitable membrane material may be used for the membrane separation. Preference is given to using an OSN (organic solvent nanofiltration) membrane material in the membrane separation after the evaporation of the process according to the invention. Such a membrane material preferably consists at least of a separation-active layer (also: active separation layer) and a substructure on which the separation-active layer is present. The membrane material of the invention preferably consists at least of a separation-active layer and a substructure.

The membrane separation after the evaporation is preferably carried out at a temperature in the range from 25°° C. to 100° C., further preferably in the range from 30° C. to 80° C. and particularly preferably in the range from 40°° C. to 70° C. To bring the product mixture to the prevailing temperature preferred for the membrane separation, the product mixture may be cooled. In addition to active cooling using a coolant, cooling may also be achieved via a heat exchanger, whereby another stream is heated within the process of the invention.

The transmembrane pressure (TMP) in the membrane separation in step c is preferably in the range from 10 to 60 bar, further preferably in the range from 15 to 55 bar, particularly preferably in the range from 40 to 50 bar. The permeate-side pressure here may be above atmospheric pressure and preferably up to 15 bar, preferably 2 to 7 bar. The difference between the TMP and the permeate-side pressure gives the retentate-side pressure. In a preferred embodiment, care should be taken, in the case of the pressure ratios and the permeate-side pressure in particular, to ensure that the pressure is set such that evaporation after passage through the membrane is avoided. Evaporation could lead to unstable operation.

In the subsequent step d, the distillative processing of the crude product mixture is carried out in at least one distillation column to remove the unreacted diisobutenes. This affords an aldehyde product comprising the aldehydes formed. The aldehyde product comprises the aldehydes formed from the diisobutene, i.e. trimethylhexanal.

In the distillative processing of the crude product mixture in step d, the unreacted diisobutenes are obtained at the top of the at least one distillation column. Aldehyde product thus accumulates in the bottoms of the at least one distillation column. The overhead stream comprising the unreacted diisobutenes removed in the at least one distillation column is recycled to the hydroformylation in step b. If the components used undergo mixing prior to the hydroformylation, the overhead stream is by definition supplied to the mixing. This allows continuous operation of the process of the invention at the highest possible yield. A purge can be withdrawn from the recycled overhead stream in order to discharge low-boiling by-products from the process.

The distillative processing to remove the unreacted diisobutenes in step d can be carried out in a distillation column. It would be conceivable for the distillative processing for the removal of the unreacted diisobutenes in step d to be carried out in more than one distillation column. This would however mean a significant higher outlay on equipment. It is therefore preferable that the distillative processing in step d is carried out in a single distillation column.

The pressure in the distillation column in the distillative processing in step d is preferably in the range from 0.3 to 2 bar, further preferably in the range from 0.4 to 1 bar, particularly preferably in the range 0.5 to 0.7 bar. The temperature in the bottoms of the distillation column in the distillative processing in step d is preferably in the range from 80° C. to 160° C. The temperature at the top of the distillation column in the distillative processing in step d is preferably in the range from 30 to 80° C. In addition, it is preferable that the reflux ratio in the distillation column is between 1 and 2. The distillation column for the removal in step d comprises preferably 10 to 30 theoretical plates. The distillation column may contain high-performance structured packings. Suitable high-performance structured packings are known to those skilled in the art.

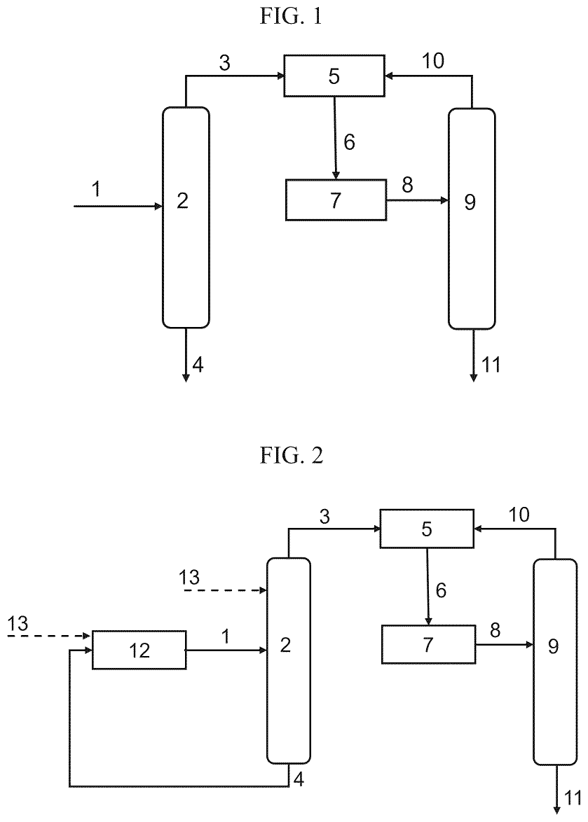

The present invention is elucidated more particularly with reference to FIG. 1 and FIG. 2. The drawings show specific embodiments and are not intended to limit the subject matter of the invention.

FIG. 1 shows an embodiment in which the diisobutene stream (1) is supplied to the distillation column (2) in step a. The overhead stream (3) is enriched with 2,4,4-trimethylpent-1-ene compared to the diisobutene stream used. The distillation also gives rise to the residual stream (4) that is depleted in 2,4,4-trimethylpent-1-ene compared to the diisobutene stream used. The overhead stream (3) is subsequently supplied to the hydroformylation (5) in step b, resulting in a product mixture (6). This product mixture (6) undergoes catalyst removal (7) in step c to afford the crude product mixture (8), which is subjected to a distillation (9) in step d. In this distillation, the product (11) and the unreacted 2,4,4-trimethylpent-2-ene and 2,4,4-trimethylpent-1-ene are obtained as a stream (10) that is recycled to the hydroformylation (5).

FIG. 2 relates to a further embodiment in which an isomerization (12) is additionally included. The employed diisobutene stream (13) can here be supplied to the isomerization and/or to the distillation (2). This is indicated by the dashed lines. The isomerized stream (1) then becomes the inflow to the distillation (2). The isomerization is also fed with the residual stream (4) from the distillation, in order there to convert 2,4,4-trimethylpent-2-ene at least partially into 2,4,4-trimethylpent-1-ene.

1. A process for the hydroformylation of diisobutenes, wherein the process comprises:
(a) supplying a diisobutene stream, comprising 2,4,4-trimethylpent-2-ene and 2,4,4-trimethylpent-1-ene, to at least one distillation column and separating the diisobutene stream into at least one overhead stream that is enriched with 2,4,4-trimethylpent-1-ene compared to the diisobutene stream used and contains more than 85% by weight of 2,4,4-trimethylpent-1-ene, and a residual stream depleted in 2,4,4-trimethylpent-1-ene compared to the diisobutene stream used;
(b) supplying the at least one overhead stream to the hydroformylation whereby 2,4,4-trimethylpent-2-ene and 2,4,4-trimethylpent-1-ene are in a reaction zone reacted with synthesis gas in the presence of a homogeneous catalyst system, comprising at least Co or Rh and optionally a phosphorus-containing ligand to obtain a liquid product mixture, the liquid product mixture comprising at least one aldehyde formed by the hydroformylation, the homogeneous catalyst system and unreacted 2,4,4-trimethylpent-2-ene and 2,4,4-trimethylpent-1-ene;
(c) removing the homogeneous catalyst system from the liquid product mixture to obtain a crude product mixture, comprising the at least one aldehyde formed by the hydroformylation and unreacted 2,4,4-trimethylpent-2-ene and 2,4,4-trimethylpent-1-ene;
(d) distillative processing of the crude product mixture in at least one distillation column to remove unreacted 2,4,4-trimethylpent-2-ene and unreacted 2,4,4-trimethylpent-1-ene, wherein the unreacted 2,4,4-trimethylpent-2-ene and 2,4,4-trimethylpent-1-ene are removed and recycled to the hydroformylation in (b).

2. The process according to claim 1, wherein the residual stream in (a) is withdrawn as a bottoms stream or as a side stream of the at least one distillation column.

3. The process according to claim 1, wherein the residual stream is supplied to an isomerization in which the 2,4,4-trimethylpent-2-ene is at least partially isomerized to 2,4,4-trimethylpent-1-ene using a heterogeneous catalyst based on a zeolite or on an ion-exchange resin.

4. The process according to claim 3, wherein the isomerization affords an isomerization stream in which a proportion of 2,4,4-trimethylpent-2-ene is lower than in the residual stream and a proportion of 2,4,4-trimethylpent-1-ene is higher than in the residual stream.

5. The process according to claim 3, wherein the isomerization stream is sent at least in part to the distillation in (a).

6. The process according to claim 3, wherein an input stream with which the process is fed and which comprises 2,4,4-trimethylpent-2-ene and 2,4,4-trimethylpent-1-ene is supplied to the distillation unit in (a) in addition to the isomerization stream and/or to the isomerization in addition to the residual stream.

3. rocess according to claim 3, wherein the isomerization is carried out at a temperature of from 25 to 90° C.

8. The process according to claim 3, wherein the zeolite has a Si:Al ratio in a range from 40:1 to 200:1.

9. The process according to claim 3, wherein the zeolite is selected from the group consisting of beta-zeolites and gamma-zeolites.

10. The process according to claim 3, wherein the ion-exchange resin used is one produced by the sulfonation of phenol/aldehyde condensates or by the sulfonation of copolymers of aromatic vinyl compounds.

11. The process according to claim 1, wherein the hydroformylation in (b) is carried out at a temperature of from 90 to 250° C.

12. The process according to claim 1, wherein the hydroformylation in (b) is carried out at a pressure of from 100 to 350 bar.

13. The process according to claim 1, wherein the removal of the homogeneous catalyst system in (c) is effected by thermal separation and/or membrane separation.

14. The process according to claim 1, wherein the removal of the homogeneous catalyst system in (c) is effected by evaporation and subsequent membrane separation.

15. The process according to claim 3, wherein the isomerization is carried out at a temperature of from 35 to 70° C.

16. The process according to claim 1, wherein the hydroformylation in (b) is carried out at a temperature of from 120 to 170° C.

17. The process according to claim 1, wherein the hydroformylation in (b) is carried out at a pressure of from 200 to 300 bar.
